(19) United States
(12) Patent Application Publication
(10) Pub. No.: US 2019/0261870 A1
(43) Pub. Date: Aug. 29, 2019

(54) BAG-SHAPED STRUCTURE AND METHOD FOR MANUFACTURING BAG-SHAPED STRUCTURE

(71) Applicants: OMRON CORPORATION, Kyoto-shi, Kyoto (JP); OMRON HEALTHCARE CO., LTD., Muko-shi, Kyoto (JP)

(72) Inventor: Kazuyoshi NISHIKAWA, Ritio-shi (JP)

(21) Appl. No.: 16/349,034

(22) PCT Filed: Dec. 26, 2017
(86) PCT No.: PCT/JP2017/046646

§ 371 Date: May 10, 2019

(30) Foreign Application Priority Data
Dec. 27, 2016 (JP) 2016-254274

(51) Int. Cl. A61B 5/022; B29C 65/04

(52) U.S. Cl. CPC A61B 5/02233; A61B 5/6824; B29C 2791/006; B29C 65/04

(57) ABSTRACT

Providing a bag-shaped structure and a method for manufacturing a bag-shaped structure, which can provide high blood pressure measurement accuracy even when the cuff width is reduced. A bag-shaped structure used in a cuff for a blood pressure monitor configured to be wrapped around a living body, inflate when a fluid is supplied to an internal space, and compress the living body includes: an inner wall portion provided on a living body side; an outer wall portion facing the inner wall portion; and a pair of side wall portions bent toward the internal space and continuous with the inner wall portion and the outer wall portion, at least part of which is formed integrally with the inner wall portion.

### FIELD

The present invention relates to a bag-shaped structure that compresses a living body in blood pressure measurement, and a method for manufacturing the bag-shaped structure.

### BACKGROUND

In recent years, a blood pressure monitor used for blood pressure measurement is utilized not only in medical facilities, but also in households as means for checking health conditions. A blood pressure monitor measures a blood pressure by detecting pulses generated in an artery and vibrations of an arterial wall, by wrapping a cuff including a bag-shaped structure around an upper arm or a wrist, etc. of a human body and by inflating and deflating the bag-shaped structure. Such a blood pressure monitor is required to have a narrower cuff for improvement in handleability and size reduction of the cuff.

Regarding the cuff used for such a blood pressure monitor, a configuration is known in which a fluid bag, which is an inflatable bag-shaped structure, is provided in a belt-shaped bag including an outer cuff piece and an inner cuff piece. Regarding the fluid bag, a technique of providing the fluid bag with: side wall portions integrally joined to an outer wall portion opposed to the outer cuff piece and an inner wall portion opposed to the inner cuff piece, and folded to the inside of the fluid bag; and a coupling portion that couples both side wall portions in the fluid bag is known (see, for example, Jpn. Pat. Appln. KOKAI Publication No. 2001-224558).

With the coupling portion that couples both side wall portions in the fluid bag, the fluid bag can maintain the shape in which both side wall portions are folded. In the fluid bag, the coupling portion couples both side wall portions; therefore, at the time of inflation, outward expansion of both side wall portions is restricted, and the cuff inflates in the thickness direction. As a result, the cuff can compress the measurement site more stably, and exhibits high compression performance. For this reason, the technique of Jpn. Pat. Appln. KOKAI Publication No. 2001-224558 can provide a cuff for a blood pressure monitor suitable for reduction in the cuff width.

### SUMMARY

In a general fluid bag of a cuff for a blood pressure monitor, an outer wall portion, an inner wall portion, and both side wall portions are joined together by laser welding, high-frequency welding, hot press welding, boding with an adhesive or double-sided tape, or the like. Therefore, the fluid bag has a joint margin on a periphery of the inner wall portion on the living body side. This joint margin does not compress the living body because compressed air in the fluid bag does not make contact with the joint margin when the fluid bag is inflated. Although the vascular compression area decreases due to the reduction in the cuff width, such a fluid bag further decreases the vascular compression area by the area of the joint margin; therefore, further reduction in the cuff width may cause deterioration of blood pressure measurement characteristics, and lower blood pressure measurement accuracy.

Therefore, the present invention is intended to provide a bag-shaped structure and a method for manufacturing a bag-shaped structure, which can provide high blood pressure measurement accuracy even when the cuff width is reduced.

According to a first aspect of the present invention, provided is a bag-shaped structure used in a cuff for blood pressure measurement configured to be wrapped around a living body, inflate when a fluid is supplied to an internal space, and compress the living body, the bag-shaped structure comprising: an inner wall portion provided on a living body side; an outer wall portion facing the inner wall portion; a pair of side wall portions bent toward the internal space and continuous with the inner wall portion and the outer wall portion, at least part of which is formed integrally with the inner wall portion.

According to a second aspect of the present invention, provided is the bag-shaped structure according to the first aspect, wherein the inner wall portion, the outer wall portion, and the pair of side wall portions are molded from a thermoplastic elastomer.

According to a third aspect of the present invention, provided is the bag-shaped structure according to the first aspect, wherein the pair of side wall portions each include: a part on the living body side with respect to an intermediate position in a direction in which the inner wall portion faces the outer wall portion, which is formed integrally with the inner wall portion; a part on another side with respect to the intermediate position in the direction in which the inner wall portion faces the outer wall portion, which is formed integrally with the outer wall portion; and a joint portion at the intermediate position where both parts are joined.

According to a fourth aspect of the present invention, provided is the bag-shaped structure according to the first aspect, wherein the inner wall portion and the pair of side wall portions are integrally formed.

According to a fifth aspect of the present invention, provided is the bag-shaped structure according to the first aspect, wherein the pair of side wall portions are each bent toward the internal space at a plurality of positions.

According to a sixth aspect of the present invention, provided is a method for manufacturing a bag-shaped structure used in a cuff for blood pressure measurement configured to be wrapped around a living body, inflate when a fluid is supplied to an internal space, and compress the living body, the bag-shaped structure comprising: an inner wall portion provided on a living body side; an outer wall portion facing the inner wall portion; and a pair of side wall portions bent toward the internal space, the method comprising: forming a thermoplastic elastomer sheet into a sheet member in a shape of the inner wall portion and at least part of each of the pair of side wall portions; trimming the formed sheet; and joining the sheet member to another sheet member in a shape of other part of the side wall portion and the outer wall portion.

According to a seventh aspect of the present invention, provided is the method for manufacturing a bag-shaped structure according to the sixth aspect, wherein the sheet member is formed by vacuum blow molding.

According to an eighth aspect of the present invention, provided is the method for manufacturing a bag-shaped structure according to the sixth aspect, wherein the sheet member and the other sheet member include joint margins for joining, and the joint margins are joined in the joining of the sheet member and the other sheet member.

According to a ninth aspect of the present invention, provided is the method for manufacturing a bag-shaped structure according to the sixth aspect, wherein the joining of the joint margins is performed by a high-frequency welder.

According to the first aspect, since the inner wall portion and at least part of the side wall portions of the bag-shaped structure are integrally formed, the joint margins are not positioned in the inner wall portion which comes into contact with the living body; therefore, high blood pressure measurement accuracy can be attained even when the cuff width is reduced.

According to the second aspect, since the inner wall portion, the outer wall portion, and the pair of side wall portions are molded from a thermoplastic elastomer, the shape is stable after the inner wall portion, the outer wall portion, and the pair of side wall portions are shaped.

According to the third aspect, since the joint margins are provided in a portion not relating to blood vessel compression, the blood vessel compression area is maximized; therefore, high blood pressure measurement accuracy can be attained even when the cuff width is reduced. In addition, when the bag-shaped structure inflates, the rigidity of the joint portions suppresses inflation of the side wall portions, that is, inflation in the non-compression direction; therefore, higher blood pressure measurement accuracy can be attained.

According to the fourth aspect, the integral formation can reduce the joint reliability risk of the joint portion, such as separation of the joint portion, caused by repeated inflations and deflations.

According to the fifth aspect, since the side wall portions are bent to the internal space at a plurality of positions, the bag-shaped structure is apt to inflate in a direction that compresses the living body, and easily follows the shape of the living body; therefore, high blood pressure measurement accuracy can be attained even when the cuff width is reduced. In addition, the bellows effect increases.

According to a sixth aspect, since a thermoplastic elastomer sheet is formed into a sheet member in a shape of the inner wall portion and at least part of each of the pair of side wall portions, the formed sheet is trimmed, and the sheet member is joined to another sheet member in a shape of other part of the side wall portion and the outer wall portion, the joint margins are not positioned in the inner wall portion which comes into contact with the living body; therefore, high blood pressure measurement accuracy can be attained even when the cuff width is reduced.

According to the seventh aspect, since the sheet member is formed by vacuum blow molding, the number of joint portions is reduced; therefore, the number of joining steps can be reduced, and cost reduction can be realized.

According to the eighth aspect, since the inner wall portion which comes into contact with the living body can be formed by a sheet member having a uniform thickness, variations in blood pressure compression characteristics resulting from non-uniform thickness of the sheet are unlikely to be caused; therefore high blood pressure measurement accuracy can be attained.

According to the ninth aspect, the joining of the joint margins is performed by a high-frequency welder; therefore, the number of processing steps can be reduced.

### BRIEF DESCRIPTION OF THE DRAWINGS

FIG. 1 is a perspective view showing a configuration of a blood pressure monitor according to one embodiment of the present invention.

FIG. 2 is a cross-sectional view showing a configuration of a cuff used in the blood pressure monitor.

FIG. 3 is a cross-sectional view schematically showing a state where the cuff is wrapped around a living body.

FIG. 4 is a cross-sectional view schematically showing a state where the cuff is inflated with the cuff wrapped around the living body.

FIG. 5 is a partially cutaway perspective view showing a configuration of a bag-shaped structure used in the cuff.

FIG. 6 is a cross-sectional view showing a configuration of the bag-shaped structure.

FIG. 7 is a flowchart showing an example of a method for manufacturing the bag-shaped structure.

FIG. 8 is an explanatory view schematically showing an example of the manufacturing method.

FIG. 9 is a partially cutaway perspective view showing a state in one process of the method for manufacturing the bag-shaped structure.

FIG. 10 is a cross-sectional view showing a main part configuration of each of the bag-shaped structure and a bag-shaped structure according to a prior art.

FIG. 11 is a cross-sectional view showing a configuration of a bag-shaped structure according to a first modification of the present invention.

FIG. 12 is a cross-sectional view showing a configuration of a bag-shaped structure according to a second modification of the present invention.

FIG. 13 is a cross-sectional view showing a configuration of another example of the bag-shaped structure according to the second modification.

FIG. 14 is a cross-sectional view showing a configuration of a bag-shaped structure according to a third modification of the present invention.

FIG. 15 is a cross-sectional view showing a configuration of a bag-shaped structure according to a fourth modification of the present invention.

FIG. 16 is a cross-sectional view showing a configuration of a bag-shaped structure according to a fifth modification of the present invention.

FIG. 17 is a cross-sectional view showing a configuration of a bag-shaped structure according to a sixth modification of the present invention.

FIG. 18 is an explanatory view showing an example of a method for manufacturing the bag-shaped structure.

### DETAILED DESCRIPTION

### First Embodiment

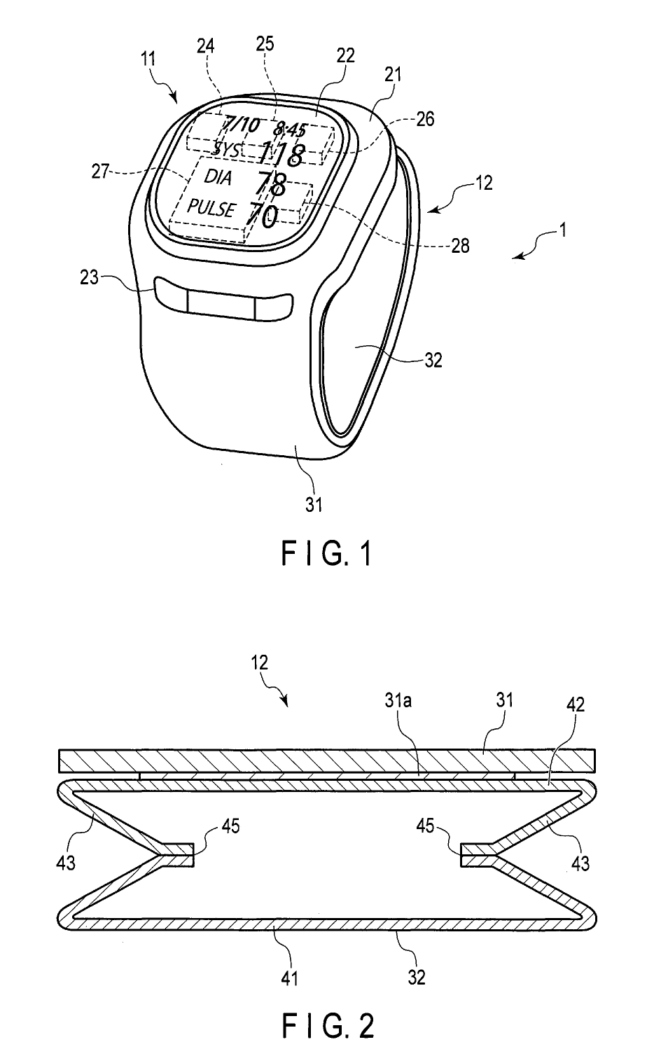

Hereinafter, a blood pressure monitor 1 including a cuff 12 with a bag-shaped structure 32 according to a first embodiment of the present invention will be described with reference to FIGS. 1 to 10.

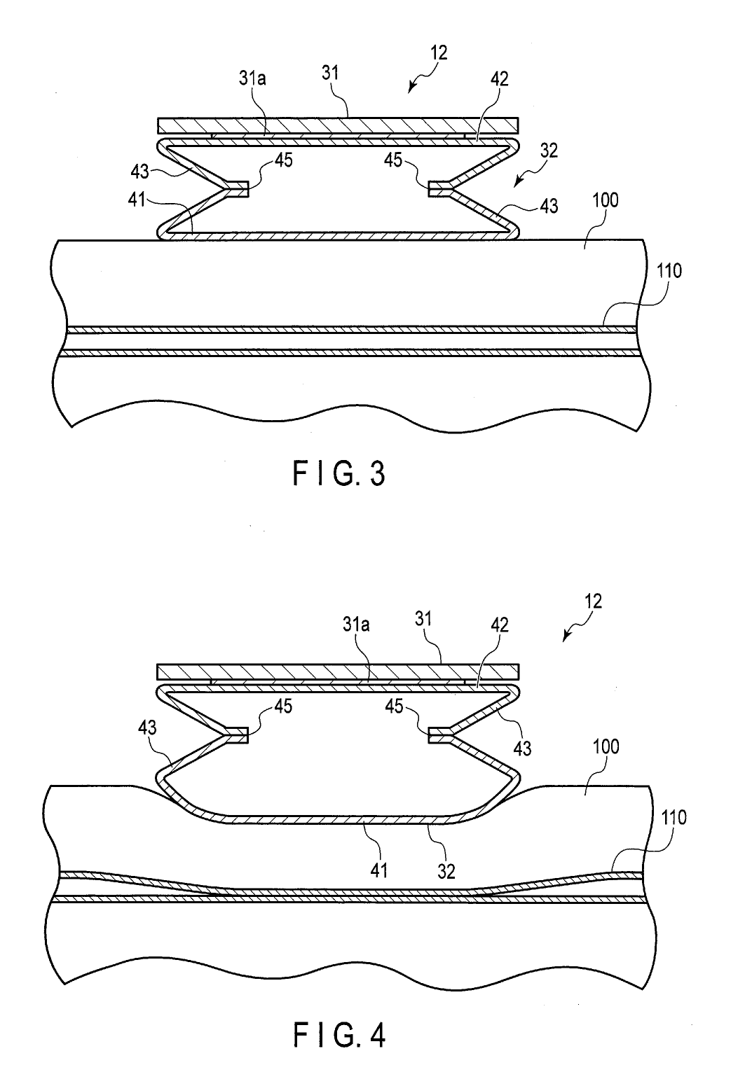

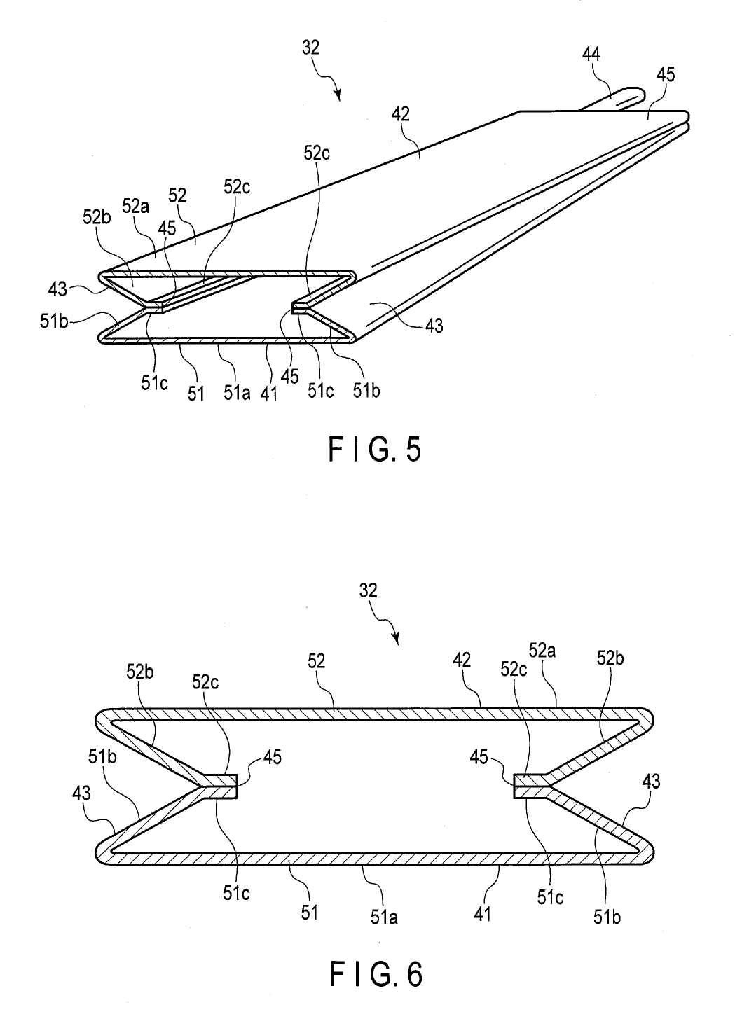

FIG. 1 is a perspective view showing a configuration of the blood pressure monitor 1 according to the first embodiment of the present invention. FIG. 2 is a cross-sectional view showing a configuration of the cuff 12 used in the blood pressure monitor 1. FIG. 3 is a cross-sectional view schematically showing a state where the cuff 12 is wrapped around a wrist 100 of a living body. FIG. 4 is a cross-sectional view schematically showing a state where the bag-shaped structure 32 of the cuff 12 is inflated with the cuff 12 wrapped around the wrist 100, and is compressing an artery 110. FIG. 5 is a partially-cutaway perspective view showing a configuration of the bag-shaped structure 32 used in the cuff 12. FIG. 6 is a cross-sectional view showing a configuration of the bag-shaped structure 32.

The blood pressure monitor 1 is an electronic blood pressure monitor to be worn on a living body, for example, the wrist 100. As shown in FIG. 1, the blood pressure monitor 1 includes an apparatus main body 11 and a cuff 12.

The apparatus main body 11 includes a case 21, a display unit 22, an operation unit 23, a pump 24, an on-off valve 25, a pressure sensor 26, a power supply unit 27, and a controller 28. The apparatus main body 11 also includes an air flow path that fluidly connects the pump 24, the on-off valve 25, the pressure sensor 26, and the cuff 12. For example, the air flow path is formed by arranging a tube or the like made of a resin material or the like in the case 21.

The display unit 22 is arranged on the upper surface of the case 21. The case 21 houses the pump 24, the on-off valve 25, the pressure sensor 26, the power supply unit 27, and the controller 28. The case 21 is connected integrally to the cuff 12.

The display unit 22 is electrically connected to the controller 28. The display unit 22 is, for example, a liquid crystal display unit or an organic electroluminescence display unit. The display unit 22 displays various information including measurements such as blood pressure values, e.g., a systolic blood pressure and a diastolic blood pressure, and a heart rate.

The operation unit 23 is configured to receive a command from the user. For example, the operation unit 23 is a button provided in the case 21, or a touch panel provided in the display unit. The operation unit 23 converts the command into an electrical signal in response to a user's operation. The operation unit 23 is electrically connected to the controller 28 and outputs the electrical signal to the controller 28.

The pump 24 is, for example, a rolling pump. The pump 24 compresses air, and supplies compressed air to the cuff 12 via the flow path. The pump 24 is electrically connected to the controller 28.

The on-off valve 25 is an electromagnetic valve electrically connected to the controller 28. The on-off valve 25 is opened and closed in accordance with the command from the controller 28. When being opened, the on-off valve 25 makes the flow path continuous with the atmosphere and reduces the pressure in the flow path.

The pressure sensor 26 detects the pressure in the flow path. The pressure sensor 26 is electrically connected to the controller 28, converts the detected pressure into an electrical signal, and outputs it to the controller 28. Here, since the flow path is continuous with the bag-shaped structure 32 (to be described later) of the cuff 12, the pressure in the flow path is equal to the pressure in the internal space of the bag-shaped structure 32.

The power supply unit 27 is, for example, a secondary battery such as a lithium ion battery. The power supply unit 27 is electrically connected to the controller 28. The power supply unit 27 supplies power to the controller 28.

The controller 28 supplies power to the display unit 22, the operation unit 23, the pump 24, the on-off valve 25, and the pressure sensor 26. The controller 28 also controls the operations of the display unit 22, the pump 24, and the on-off valve 25 based on the electrical signals output from the operation unit 23 and the pressure sensor 26.

For example, when a command to measure a blood pressure is input from the operation unit 23, the controller 28 drives the pump 24 and sends compressed air to the cuff 12. Furthermore, the controller 28 controls whether to drive or stop the pump 24 and whether to open or close the on-off valve 25 based on the electrical signal output by the pressure sensor 26. Moreover, the controller 28 obtains measurements such as a blood pressure values, e.g., a systolic blood pressure or a diastolic blood pressure, and a heart rate from the electrical signal output by the pressure sensor 26, and outputs an image signal corresponding to the measurements to the display unit 22.

As shown in FIGS. 1 and 2, the cuff 12 includes a base material 31 and a bag-shaped structure 32. The cuff 12 is fixed to the wrist by being wrapped around the wrist.

The base material 31 is curved along the shape of the arm. For example, the base material 31 has one end formed integrally with the case 21, and the other end fixable to the case 21 with a fastener or the like. The base material 31 supports the bag-shaped structure 32 on its inner surface. For example, the base material 31 includes, on its inner surface, a joint layer 31a such as an adhesive or a double-sided tape for joining the bag-shaped structure 32 thereto. The base material 31 is made of a hard resin material.

As shown in FIGS. 2 to 6, the bag-shaped structure 32 includes: a rectangular inner wall portion 41 elongated in one direction; a rectangular outer wall portion 42 elongated in one direction; a pair of side wall portions 43 joining the inner wall portion 41 and the outer wall portion 42 and bending toward the inside of the bag-shaped structure 32; and a connection tube 44 fluidly connecting an inner space formed by the inner wall portion 41, the outer wall portion 42, and the pair of side wall portions 43 to the flow path of the apparatus main body 11.

In the bag-shaped structure 32, an air chamber fluidly connected to the flow path of the apparatus main body 11 is formed by the inner wall portion 41, the outer wall portion 42, and the pair of side wall portions 43. The bag-shaped structure 32 is arranged on the base material 31 so as to be curved along the inner surface of the base material 31. The width of the bag-shaped structure 32 is set to, for example, 40 mm or less. Such a bag-shaped structure 32 is sometimes called a structure because the side wall portions 43 bend toward the inside of the bag-shaped structure 32.

In the bag-shaped structure 32, the inner wall portion 41 and at least part of the pair of side wall portions 43 are integrated. As shown in FIG. 5, both longitudinal ends of the bag-shaped structure 32 are closed by joining the inner wall portion 41 and the outer wall portion 42 at both longitudinal ends. The bag-shaped structure 32 is formed by, for example, integrally joining a plurality of sheet members each having a predetermined shape and made of a thermoplastic elastomer.

As the thermoplastic elastomer constituting each sheet member, for example, thermoplastic polyurethane resin (hereinafter referred to as TPU), polyvinyl chloride resin, ethylene-vinyl acetate resin, thermoplastic polystyrene resin, thermoplastic polyolefin resin, thermoplastic polyester resin, or thermoplastic polyamide resin can be used. As the thermoplastic elastomer, TPU is preferably used. Furthermore, the sheet member may have a single-layer structure or may have a multi-layer structure.

In the present embodiment, the bag-shaped structure 32 includes a first sheet member 51 constituting the inner wall portion 41 and part of the pair of side wall portions 43, and a second sheet member 52 constituting the outer wall portion 42 and the other part of the pair of side wall portions 43. The bag-shaped structure 32 is formed by joining the first sheet member 51 and the second sheet member 52. The bag-shaped structure 32 includes joint portions 45, where the first sheet member 51 and the second sheet member 52 are joined, in a center portion of the side wall portions 43 and on the inner surface side of the bag-shaped structure 32. Each of the joint portions 45 is a portion where the sheet members 51 and 52, etc. are joined by welding.

As shown in FIGS. 5 and 6, the first sheet member 51 includes a rectangular first portion 51a constituting the inner wall portion 41, a pair of rectangular second portions 51b constituting halves of the side wall portions 43 with respect to the height direction of the side wall portions 43, and joint margins 51c provided at ends of the second portion 51b. Here, the height direction of the side wall portions 43 refers to the direction in which the inner wall portion 41 faces the outer wall portion 42. The longitudinal length of the first sheet member 51 is equal to the longitudinal length of the bag-shaped structure 32.

Regarding the first sheet member 51, the first portion 51a and the second portion 51b are formed in advance into the shape of the inner wall portion 41 and the halves of the pair of side wall portions 43 by vacuum blow molding.

As shown in FIGS. 5 and 6, the second sheet member 52 includes a rectangular first portion 52a constituting the outer wall portion 42, a pair of rectangular second portions 52b constituting halves of the side wall portions 43 with respect to the height direction, and joint margins 52c provided at ends of the second portion 52b. The longitudinal length of the second sheet member 52 is equal to the longitudinal length of the bag-shaped structure 32.

Regarding the second sheet member 52, the first portion 52a and the second portion 52b are formed into the shape of the outer wall portion 42 and halves of the pair of side wall portions 43 by vacuum blow molding.

The first sheet member 51 and the second sheet member 52 are joined by joining the joint margins 51c and 52c and both longitudinal ends by laser welding, high-frequency welding, hot press welding, bonding with an adhesive or a double-sided tape, or the like. In addition, the connection tube 44 is fixed between one ends of the first sheet member 51 and the second sheet member 52.

The connection tube 44 is made of, for example, a resin material and is flexible. The connection tube 44 is fixed to one longitudinal end of the bag-shaped structure 32. One end of the connection tube 44 is connected to the internal space of the bag-shaped structure 32 constituted by the first sheet member 51 and the second sheet member 52. The connection tube 44 is connected to the flow path of the apparatus main body 11.

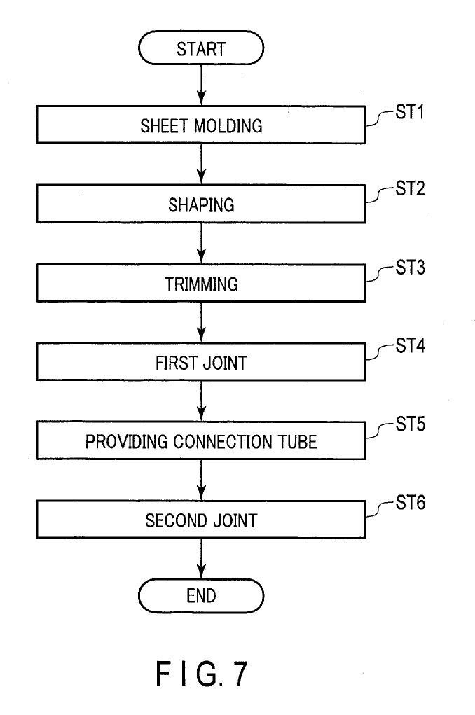

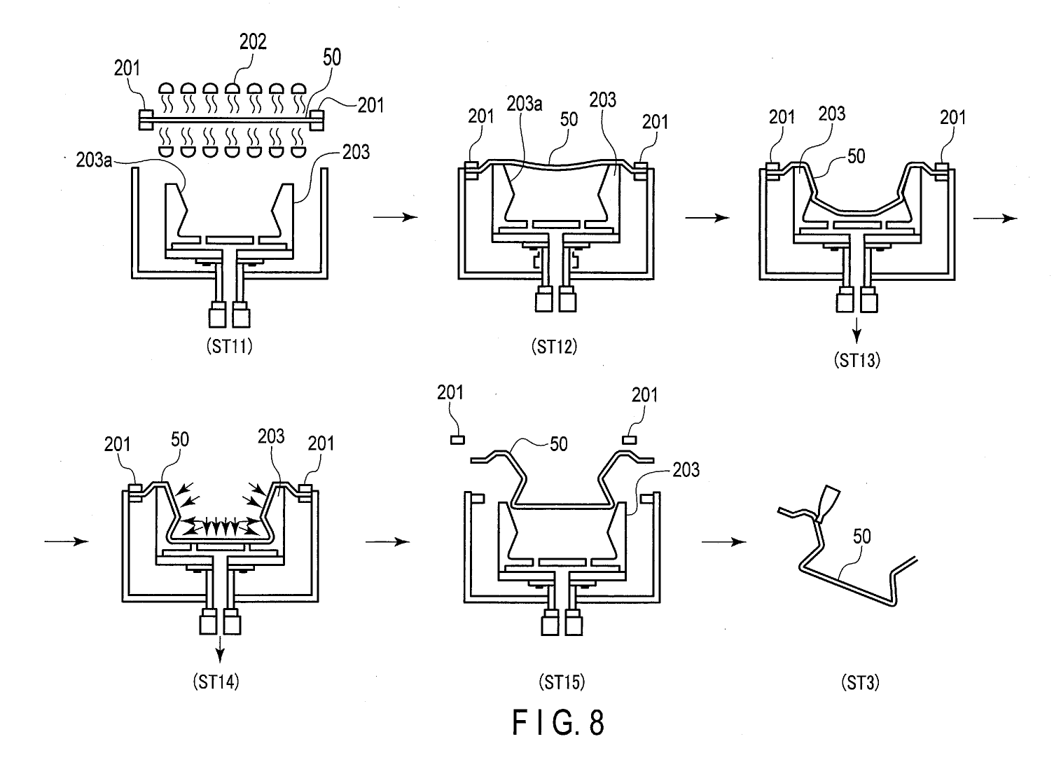

Next, a method for manufacturing the bag-shaped structure 32 will be described with reference to FIGS. 7 and 8. FIG. 7 is a flowchart showing an example of the method for manufacturing the bag-shaped structure 32. FIG. 8 is a flowchart showing an example of shaping and trimming in the method for manufacturing the bag-shaped structure 32 by schematic views.

First, a sheet 50 made of a thermoplastic elastomer is molded by T die extrusion or the like (step ST1). At this time, the sheet 50 is molded to have a rectangular shape and to be slightly thicker than the first sheet member 51 and second sheet member 52, which are molded in a process to be described later.

Next, the molded sheet 50 is formed into the shape of each of the first sheet member 51 and the second sheet member 52 by vacuum blow molding (step ST2). Specifically, first, the thermoplastic elastomer sheet 50 is held by a clamp 201 and heated by a heater 202, as shown in FIG. 8 (step ST11). Next, the sheet 50 is placed on a mold 203 having a cavity 203a in the shape of the inner wall portion 41 or outer wall portion 42 and at least part of the pair of side wall portions 43 (step ST12). At this time, the sheet 50 is brought into close contact with the mold 203 so as to cover the cavity 203a.

Next, the air between the mold 203 and the sheet 50 is vacuumed with a vacuum pump (step ST13). As a result, the thermoplastic elastomer sheet 50 heated and softened by the heater 202 is brought into close contact with the inner surface of the cavity 203a of the mold 203. The vacuuming is continued, and the sheet 50 is cooled (step ST14). By being cooled, the sheet 50 is formed into the inner surface shape of the cavity 203a of the mold 203.

As a result, the sheet 50 has the shape of the first sheet member 51 or the second sheet member 52. The thickness of each portion of the sheet 50 formed into the shape of the first sheet member 51 or the second sheet member 52 is preferably in the range of 0.05 mm to 0.50 mm, and more preferably in the range of 10 mm to 0.40 mm. This is because, if the bag-shaped structure 32 is too thin, there is a risk of tearing or the like, and if the bag-shaped structure 32 is too thick, there is a risk that shape conformability of when the bag-shaped structure 32 is inflated is deteriorated. Next, the sheet 50 is released and taken out from the mold 203 (step ST15).

Next, the sheet 50 formed into the shape of each of the first sheet member 51 and the second sheet member 52 is trimmed and finished (step ST3). Accordingly, the first sheet member 51 and the second sheet member 52 are molded.

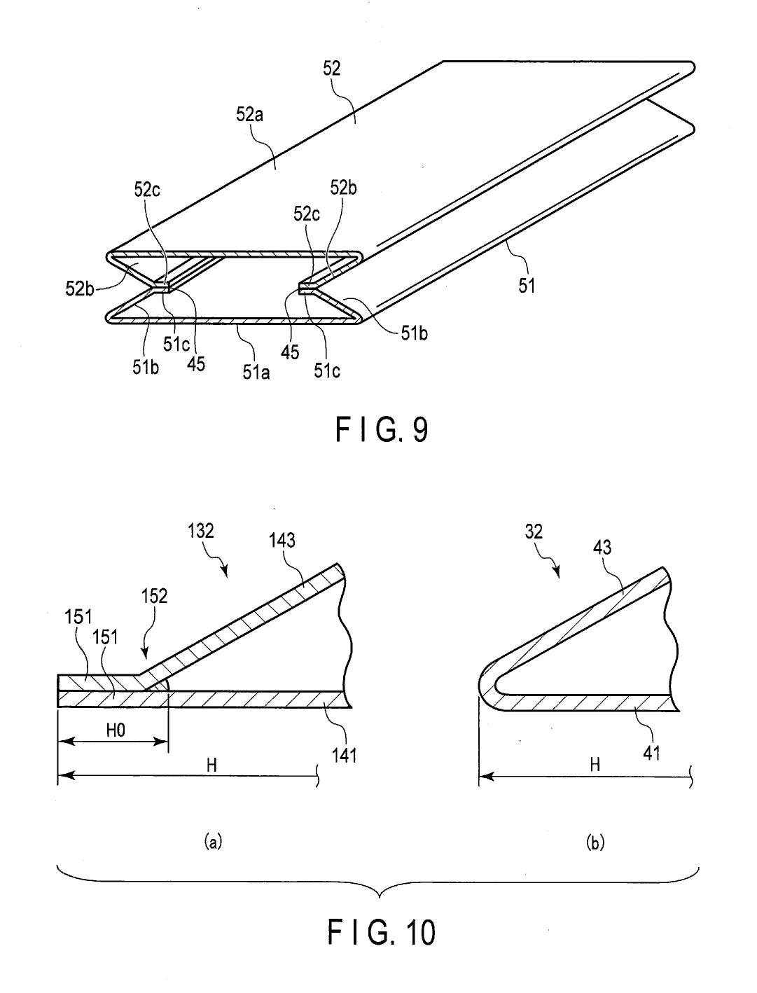

Next, as shown in FIG. 9, the first sheet member 51 and the second sheet member 52 are joined as first joining (step ST4). The joining of the first sheet member 51 and the second sheet member 52 is performed by joining the joint margins 51c and 52c by laser welding, high-frequency welding, hot press welding, bonding with an adhesive or a double-sided tape, or the like.

Next, the connection tube 44 is provided between the first sheet member 51 and the second sheet member 52 at one longitudinal ends of the first sheet member 51 and the second sheet member 52 (step ST5). Next, as shown in FIG. 5, both longitudinal ends of the first sheet member 51 and the second sheet member 52 are joined by laser welding, high-frequency welding, hot press welding, bonding with an adhesive or a double-sided tape, or the like, as second joining (step ST6).

The bag-shaped structure 32 is manufactured by those steps. The manufactured bag-shaped structure 32 is joined to the inner surface of the base material 31 via the joint layer 31a, and the connection tube 44 is fluidly connected to the flow path of the apparatus main body 11. The blood pressure monitor 1 including the cuff 12 is accordingly manufactured.

Next, measurement of a blood pressure value using the blood pressure monitor 1 will be described with reference to FIGS. 1, 3, and 4.

When measuring a blood pressure value, the user wears the cuff 12 on the living body, which is the wrist 100 in the present embodiment. As a result, the bag-shaped structure 32 of the cuff 12 comes into contact with the wrist 100, as shown in FIG. 3. Next, the user operates the operation unit 23 shown in FIG. 1 and inputs a command corresponding to the start of measurement of a blood pressure value.

The operation unit 23 to which the command input operation has been performed outputs an electrical signal corresponding to the start of measurement to the controller 28. Upon receipt of the electrical signal, the controller 28 closes the on-off valve 25, drives the pump 24, and supplies compressed air to the bag-shaped structure 32 via the flow path. As a result, the bag-shaped structure 32 starts to inflate.

The pressure sensor 26 detects the pressure in the internal space of the bag-shaped structure 32, and outputs an electrical signal corresponding to the pressure to the controller 28. Based on the received electrical signal, the controller 28 determines whether the pressure in the internal space of the bag-shaped structure 32 has reached a predetermined pressure for blood pressure measurement. When the pressure in the internal space of the bag-shaped structure 32 reaches the predetermined pressure, the controller 28 stops driving the pump 24. At this time, as shown in FIG. 4, the bag-shaped structure 32 is sufficiently inflated, and the inflated bag-shaped structure 32 compresses the wrist and blocks the artery 110 in the wrist 100.

Thereafter, the controller 28 controls the on-off valve 25 to repeatedly open and close the on-off valve 25 or adjust the opening degree of the on-off valve 25 to reduce the pressure in the internal space of the bag-shaped structure 32. Based on the electrical signal output by the pressure sensor 26 in this process of reducing the pressure, the controller 28 obtains measurements such as blood pressure values, e.g., a systolic blood pressure and a diastolic blood pressure, a heart rate, and the like. The controller 28 outputs an image signal corresponding to the obtained measurements to the display unit 22.

Upon receipt of the image signal, the display unit 22 displays the measurements on the screen. The user visually recognizes the display unit 22 to confirm the measurements. After the measurement is completed, the user unfastens the fastener and removes the blood pressure monitor 1 from the wrist.

For the cuff 12 used in the blood pressure monitor 1 according to one embodiment, which is configured as described above, the inner wall portion 41, which is provided on the living body side and is brought into contact with the human skin, is formed integrally with at least part of the side wall portions 43. That is, the cuff 12 has a configuration in which the joint margins are provided away from the site relating to vascular compression. Consequently, when the bag-shaped structure 32 is inflated, the compressed air exerts pressure on the entire inner wall portion 41, so that the inner wall portion 41 is brought into contact with the living body, and the compression area to compress the living body increases, thereby enabling uniform application of pressure by inflation. As a result, even when the cuff width is reduced, the cuff 12 can provide high blood pressure measurement accuracy.

Specifically, as in the case of the bag-shaped structure 132 of the prior art shown in FIG. 10(a), when a joint portion 152 including a joint margin 151 is provided in an inner wall portion 141 and a side wall portion 143, the second moment of area of the joint portion 152 increases, and the inner wall 141 becomes difficult to expand upon inflation. Furthermore, as shown in FIG. 10(a), the cuff width H of the bag-shaped structure 132 is equal to the distance between the ends of the joint margins 151 with respect to the width direction of the bag-shaped structure 32; therefore, the compression area decreases according to the width HO of the joint margins 151 on both sides with respect to the width direction.

In contrast, as shown in FIG. 10(b), according to the bag-shaped structure 32 of the present embodiment, the inner wall portion 41 and the side wall portions 43 are integrally formed by the second sheet member 52; therefore, the inner wall portion 41 does not include the joint portion 152. Therefore, the second moment of area in the vicinity of the boundary between the inner wall portion 41 and each side wall portion 43 is smaller than when the inner wall portion 41 includes the joint portion 152, and the compression area becomes the entire lower surface of the inner wall portion 41. Consequently, the bag-shaped structure 32 is easily inflated, and a sufficient compression area can be secured. As a result, a blood vessel can be effectively compressed, and good blood pressure measurement characteristics can be attained.

In addition, when the bag-shaped structure 32 inflates, the cuff 12 can suppress inflation of the side wall portions 43, that is, inflation in the non-compression direction, due to the rigidity of the joint portions 54 of the side wall portions 43; therefore, higher blood pressure measurement accuracy can be attained.

Furthermore, the first sheet member 51 and the second sheet member 52 are molded by vacuum blow molding and then the joint margins 51c and 52c are joined; therefore, the number of joint portions 45 can be reduced. Moreover, remnants or the like are less likely to be produced by trimming or the like at the time of molding, which prevents waste of materials. In addition, the first sheet member 51 and the second sheet member 52 are molded from a thermoplastic elastomer by using vacuum blow molding; therefore, the shape is stable after shaping the inner wall portion 41, the outer wall portion 42, and the pair of side wall portions 43.

As a result, the processing cost and the manufacturing cost of the bag-shaped structure 32 can be reduced. Furthermore, the reduction in the number of joint portions 45 reduces the risk of causing separation of each joint portion 45 by repeated inflation and deflation of the bag-shaped structure 32, and stabilizes the quality of the bag-shaped structure 32. Use of the cuff 12 using the bag-shaped structure 32 as described above enables the blood pressure monitor 1 to have improved reliability in both blood pressure measurement and quality aspects.

[Modifications]

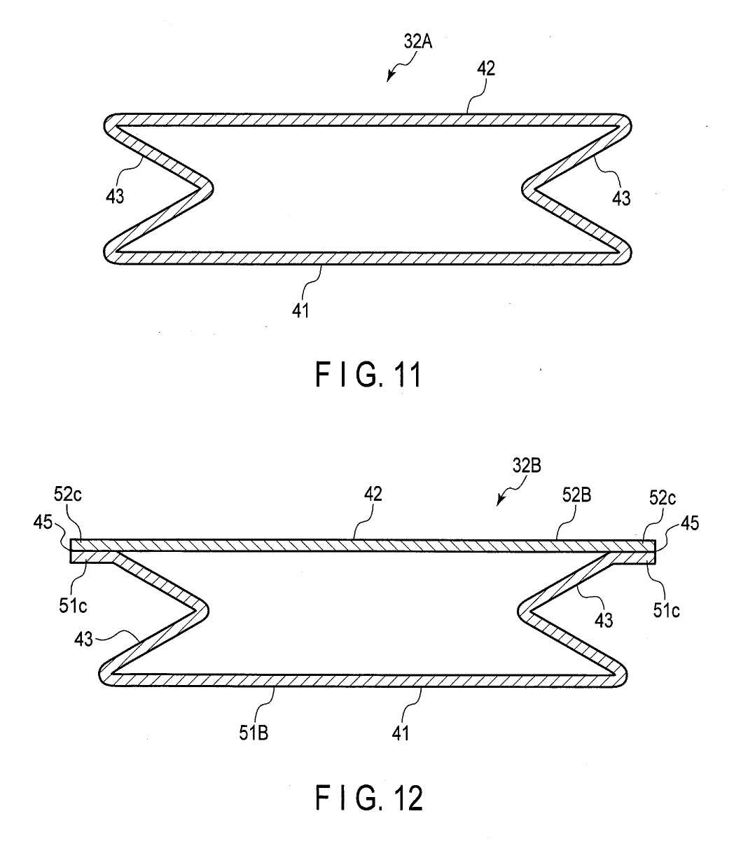

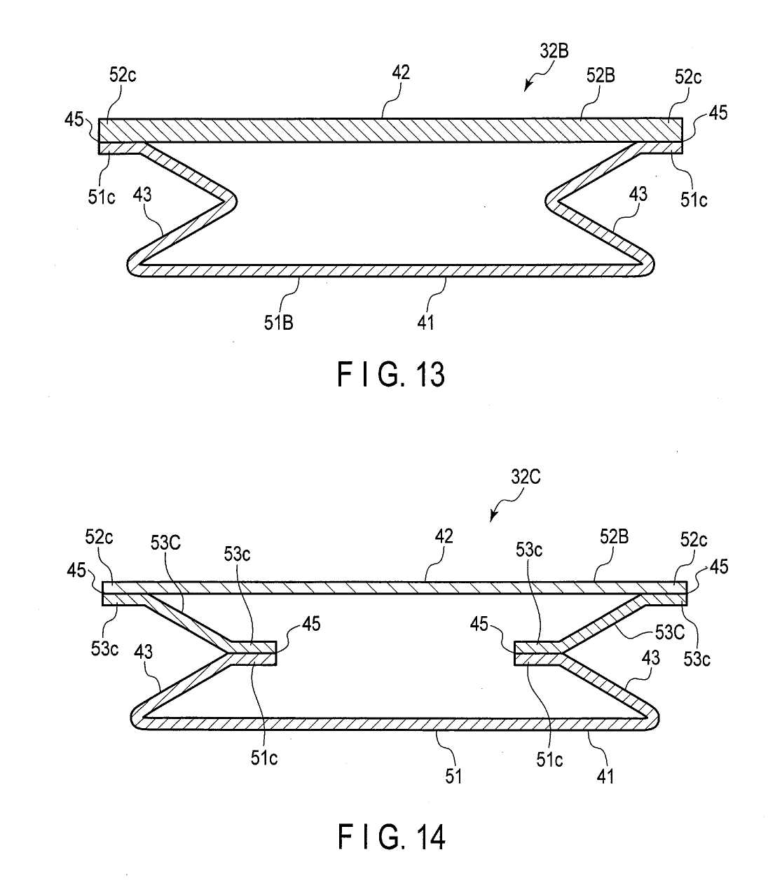

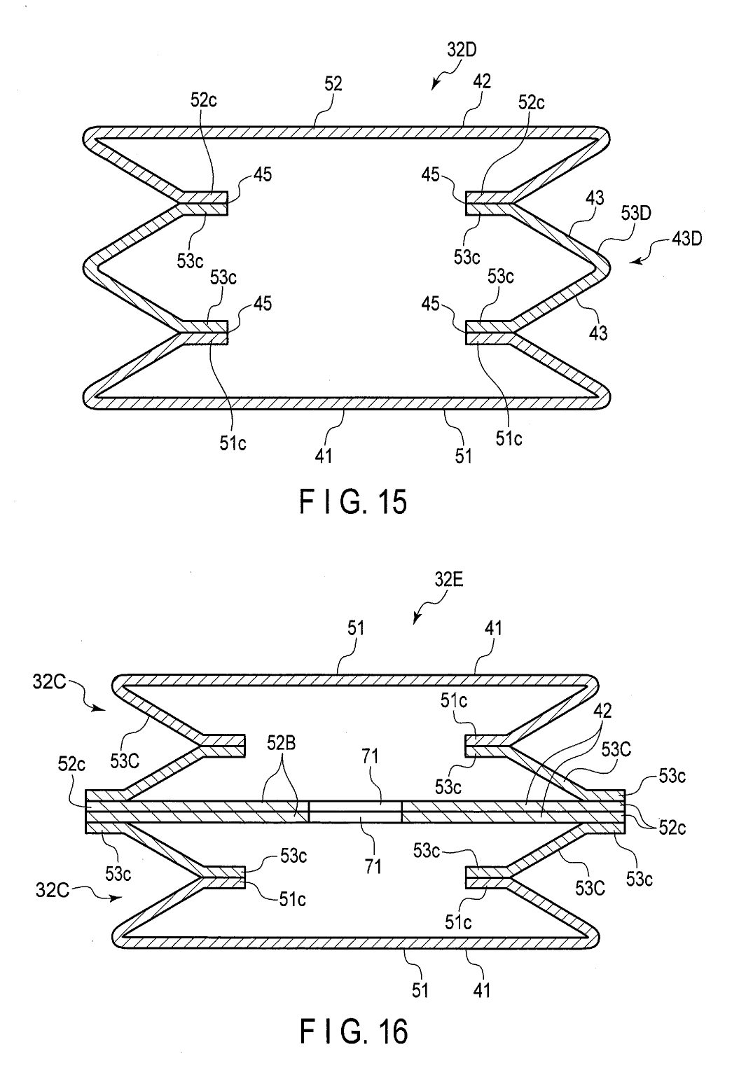

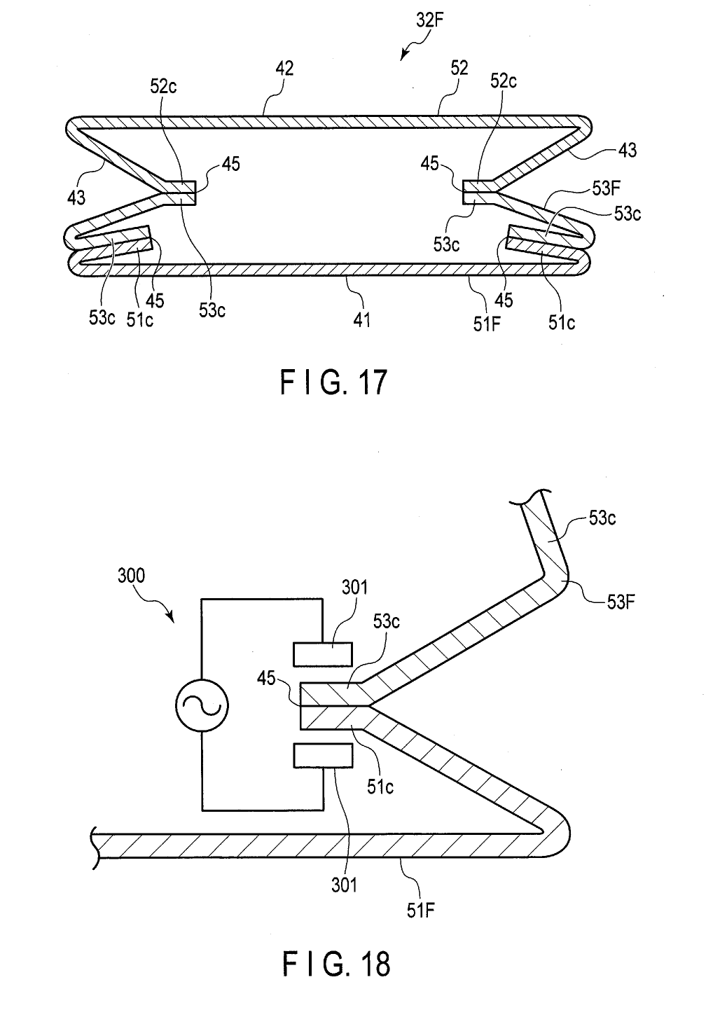

Next, modifications of the bag-shaped structure 32 will be described with reference to FIGS. 11 to 18. FIG. 11 is a cross-sectional view showing a configuration of a bag-shaped structure 32A according to a first modification. FIG. 12 is a cross-sectional view showing a configuration of a bag-shaped structure 32B according to a second modification. FIG. 13 is a cross-sectional view showing a configuration of another example of the bag-shaped structure 32B according to the second modification. FIG. 14 is a cross-sectional view showing a configuration of a bag-shaped structure 32C according to a third modification. FIG. 15 is a cross-sectional view showing a configuration of a bag-shaped structure 32D according to a fourth modification. FIG. 16 is a cross-sectional view showing a configuration of a bag-shaped structure 32E according to a fifth modification. FIG. 17 is a cross-sectional view showing a configuration of a bag-shaped structure 32F according to a sixth modification. FIG. 18 is an explanatory diagram showing an example of a method for manufacturing bag-shaped structure 32F.

[First Modification]

As shown in FIG. 11, in the bag-shaped structure 32A according to the first modification, the inner wall portion 41, the outer wall portion 42, and the pair of side wall portions 43 are integrally formed. Such a bag-shaped structure 32 is formed from one thermoplastic elastomer sheet by vacuum blow molding or the like.

For example, it is possible to: set one sheet on a mold having a cavity in the shape of the inner wall portion 41, the outer wall portion 42, and the pair of side wall portions 43; form the sheet into a shape in which only one side wall portion 43 and the outer wall portion 42 are separated from each other; and then join the outer wall portion 42 and one side wall portion 43 so as to form bag-shaped structure 32A. It is also possible to form a sheet into a shape in which the outer wall portion 42 is divided into two, and then join the divisions of the outer wall portion 42.

Like the above-described bag-shaped structure 32, when used in the cuff 12, the bag-shaped structure 32A configured as described above can provide high blood pressure measurement accuracy even when the cuff width is reduced.

[Second Modification]

As shown in FIG. 12, in the bag-shaped structure 32B according to the second modification, the inner wall portion 41 and the pair of side wall portions 43 are integrally formed, and the pair of side wall portions 43 and the outer wall portion 42 are integrated by joining. Such bag-shaped structure 32B is manufactured by joining a first sheet member 51B and a second sheet member 52B.

The first sheet member 51B is formed by molding one thermoplastic elastomer sheet into the shape of the inner wall portion 41, the pair of side wall portions 43, and the joint margins 51c by vacuum blow molding or the like. The second sheet member 52B is formed by molding one thermoplastic elastomer sheet into the shape of the outer wall portion 42 and the joint margins 52c by T die extrusion or the like. The bag-shaped structure 32B is manufactured by joining the first sheet member 51B and the second sheet member 52B by joining the joint margins 51c and 52c together.

Like the above-described bag-shaped structure 32, when used in the cuff 12, the bag-shaped structure 32A configured as described above can provide high blood pressure measurement accuracy even when the cuff width is reduced.

As shown in, for example, FIG. 13, the bag-shaped structure 32B may have a configuration in which the outer wall portion 42 is thicker than the inner wall portion 41 or the pair of side wall portions 43. This is because the outer wall portion 42 is supported by the base material 31, and thus need not be deformed in the inflation direction when the bag-shaped structure 32 is inflated. This is also because suppression in the deformation amount of the outer wall portion 42 enables further deformation of the inner wall portion 41 which comes into contact with the living body.

For the same reason, the bag-shaped structure 32B may use materials having different physical properties as the thermoplastic elastomers forming the first sheet member 51B and the second sheet member 52B constituting the outer wall portion 42. For example, deformation of the outer wall portion 42 can be suppressed by using, for the second sheet member 52B, a thermoplastic elastomer having a higher Shore A hardness than that of the first sheet member 51B.

As a method for using materials having different physical properties for the first sheet material 51B and the second sheet member 52B, the same type of materials having different physical properties may be used, or different types of materials may be used.

[Third Modification]

As shown in FIG. 14, in the bag-shaped structure 32C according to the third modification, the inner wall portion 41 and halves of the pair of side wall portions 43 with respect to the height direction are integrally formed, and the other halves of the side wall portions 43 with respect to the height direction and the outer wall portion 42 are integrated by joining. Such a bag-shaped structure 32C is manufactured by joining first sheet member 51, second sheet member 52B, and a pair of third sheet members 53C.

The first sheet member 51 has the same configuration as that of the first sheet member 51 constituting the bag-shaped structure 32 of the above-described embodiment, and the second sheet member 52B has the same configuration as that of the second sheet member 52B constituting the bag-shaped structure 32B of the above-described second modification. Each of the third sheet members 53C has the shape of the side wall portion 43 from the center to the outer wall portion 42 with respect to the height direction, and includes a pair of joint margins 53c. The third sheet member 53C is formed by molding one thermoplastic elastomer sheet into the shape of part of the side wall portions 43 and a pair of joint margins 53c, for example, by T die extrusion or the like.

The bag-shaped structure 32C is manufactured by joining the first sheet member 51, the second sheet member 52B, and the third sheet members 53C by joining joint margins 51c and 53c together and joining joint margins 52c and 53c together.

Like the above-described bag-shaped structure 32, when used in the cuff 12, the bag-shaped structure 32C configured as described above can provide high blood pressure measurement accuracy even when the cuff width is reduced.

[Fourth Modification]

As shown in FIG. 15, the bag-shaped structure 32D according to the fourth modification includes the inner wall portion 41, the outer wall portion 42, and a pair of multi-tier side wall portions 43D. For example, the multi-tier side wall portions 43D are each configured by integrally joining two side wall portions 43 in the height direction.

Such a bag-shaped structure 32D is formed by integrally forming the inner wall portion 41 and halves of a pair of side wall portions 43 with respect to the height direction, integrally forming the outer wall portion 42 and halves of a pair of side wall portions 43 with respect to the height direction, integrally forming the other halves of the two pairs of side wall portions 43 with respect to the height direction, and integrally joining them. Such a bag-shaped structure 32D is manufactured by joining first sheet member 51, second sheet member 52, and a pair of third sheet members 53D. The bag-shaped structure 32D is called a so-called bellows structure.

The first sheet member 51 and second sheet member 52 have the same configurations as the first sheet member 51 and second sheet member 52 constituting the bag-shaped structure 32 of the above-described embodiment. The third sheet member 53D is formed into the shape of the middle halves of the two side wall portions 43, and includes the pair of joint margins 53c. For example, the third sheet member 53C is formed by molding one thermoplastic elastomer sheet into the shape of the middle halves of the two side wall portions 43 with respect to the height direction and the pair of joint margins 53c by vacuum blow molding.

The bag-shaped structure 32D is manufactured by joining the first sheet member 51, the second sheet member 52, and the third sheet member 53D by joining joint margins 51c and 53c together and joining joint margins 52c and 53c together.

Like the above-described bag-shaped structure 32, when used in the cuff 12, the bag-shaped structure 32D configured as described above can provide high blood pressure measurement accuracy even when the cuff width is reduced.

In addition, since the side wall portion 43D is folded into the internal space at a plurality of positions, the bag-shaped structure 32D is apt to inflate in a direction that compresses the living body, and therefore easily follows the shape of the living body. Accordingly, even when the cuff width is reduced, the bag-shaped structure 32D can provide high blood pressure measurement accuracy, and an increased bellows effect.

[Fifth Modification]

As shown in FIG. 16, the bag-shaped structure 32E according to the fifth modification is formed by joining the outer wall portions 42 of two bag-shaped structures 32C. Specifically, the bag-shaped structure 32E includes two bag-shaped structures 32C, and a communication hole 71 which is provided in the joined two outer wall portions 42 and enables fluid communication. The communication hole 71 is provided, for example, when the second sheet member 52B is molded. In addition, in the bag-shaped structure 32E, the inner wall portion 41 of one of bag-shaped structures 32C is arranged on the living body side, and the inner wall portion 41 of the other one of the bag-shaped structures 32C is supported by the base material 31.

Like the above-described bag-shaped structure 32, when used in the cuff 12, the bag-shaped structure 32E configured as described above can provide high blood pressure measurement accuracy even when the cuff width is reduced. Furthermore, in the bag-shaped structure 32E, the outer wall portions 42 of the two bag-shaped structures 32C are joined together, and the internal spaces of the two bag-shaped structures 32C fluidly communicate with each other by the communication hole 71. Therefore, when the bag-shaped structure 32E is inflated, the blood vessel compression characteristics can be improved. In addition, since the side wall portions 43 are each deformed between the inner wall portion 41 and the outer wall portion 42, the side wall portions 43 can be prevented from being expanded and deformed outward as compared with the bag-shaped structure D of the fourth modification; therefore, the bag-shaped structure 32E can inflate more in the height direction and can provide high blood pressure measurement accuracy.

[Sixth Modification]

As shown in FIG. 17, in the bag-shaped structure 32F according to the sixth modification, the inner wall portion 41 and halves of the pair of side wall portions 43 with respect to the height direction are integrally formed by being joined at ends of the side wall portions 43, and the outer wall portion 42 and the other halves of the pair of side wall portions 43 with respect to the height direction are integrally molded. Such a bag-shaped structure 32F is manufactured by joining first sheet member 51F, second sheet member 52, and a pair of third sheet members 53F.

The first sheet member 51F is formed into the shape of the inner wall portion 41, and the ends of the inner wall portion 41 and the ends of the side wall portions 43 on the inner wall portion 41 side, and includes a pair of joint margins 51c. The second sheet member 52 has the same configuration as the second sheet member 52 constituting the bag-shaped structure 32 of the above-described one embodiment.

Each of the third sheet members 53F has the shape of the side wall portion 43 from the center to the outer wall portion 42 with respect to the height direction, and includes a pair of joint margins 53c. The third sheet member 53F is formed by molding one thermoplastic elastomer sheet into the shape of part of the side wall portion 43 and the pair of joint margins 53c by, for example, T die extrusion or the like.

The bag-shaped structure 32F is manufactured by joining the joint margins 51c and 53c of the first sheet member 51F and the third sheet member 53F and joining the joint margins 52c and 53c of the second sheet member 52 and the third sheet member 53F.

For example, joint margins 51c and 53c are joined by high-frequency welder welding as shown in FIG. 18. Specifically, joint margins 51c and 53c are folded toward the inside of the bag-shaped structure 32 to overlap one another. Then, joint margins 51c and 53c are placed between a pair of electrodes 301 of a high-frequency welder device 300 so that joint margins 51c and 53c are welded.

Next, the joint margins 52c and 53c of the second sheet member 52 and the third sheet member 53F are joined by high-frequency welder welding. The bag-shaped structure 32F is manufactured by those steps.

Like the above-described bag-shaped structure 32, when used in the cuff 12, the bag-shaped structure 32F configured as described above can provide high blood pressure measurement accuracy even when the cuff width is reduced, because the joint margins and joint portions 45 are not provided on the surface of the inner wall portion 41 which comes into contact with the living body.

Furthermore, in the bag-shaped structure 32F, ridges of the inner wall portion 41 and ridges of the side wall portions 43 on the lower end side are provided in the first sheet member 51F, and joint margins 53c are provided on the ridges. In the bag-shaped structure F, the inner wall portion 41 and the ridges of the inner wall portion 41 and the inner wall portion 41 are deformed due to inflation by compressed air. In addition, the secondary moment of area of the inner wall portion 41 does not increase, but the secondary moment of area of the side wall portions 43 increases. As a result, the lateral expansion of the side wall portions 43 is suppressed, and the bag-shaped structure 32F inflates more at the inner wall portion 41, thereby improving blood vessel compression characteristics.

The present invention is not limited to the above-described embodiment and modifications. For example, the bag-shaped structure 32 may be designed as appropriate as long as the joint portions 45 and joint margins are not provided in the inner wall portion 41 which comes into contact with the living body, and the bag-shaped structure 32 includes the side wall portions 43 which are continuous with the inner wall portion 41 and the outer wall portion 42 and bend toward the inside of the bag-shaped structure 32. For example, although the configuration in which the joint portions 45 are provided in the side wall portions 43 above the ledges of the inner wall portion 41 and the side wall portions 43 of the bag-shaped structure 32F according to the sixth modification is described, the configuration is not limited to this, and may be a configuration in which the joint portions 45 are also provided in the side wall portions 43 on the outer wall portion 42 side. Furthermore, the bag-shaped structure 32 may have a configuration in which the second moment of area is increased or decreased as appropriate by making the thickness of each portion, such as the corner of the outer wall portion 42 and the side wall portion 43, or the distal end of the side wall portion 43, different from the thickness of another portion, or a configuration in which a portion in a shape that improves, for example, the folding characteristics of the side wall portions 43 is provided.

The present invention is not limited to the above-described embodiment, and can be modified in practice, without departing from the gist of the invention. In addition, embodiments may be combined as appropriate where possible, in which case a combined advantage can be attained. Furthermore, the above-described embodiments include various stages of the invention, and various inventions can be extracted by suitably combining the structural elements disclosed herein. For example, even if some structural elements of all the structural elements disclosed in the embodiments are deleted, the embodiment from which those structural elements are deleted can be extracted as an invention as long as the problem to be solved by the invention can be solved, and the advantages of the invention can be attained.

### Examples

In order to concretize the features of the present invention, examples and evaluation tests will be described below. However, the scope of the present invention is not limited to the following examples.

As an example, the bag-shaped structure 32 of one embodiment was prepared. Thermoplastic polyurethane was used as the thermoplastic elastomer constituting the bag-shaped structure 32. The width H of the bag-shaped structure 32 was 25 mm. Since the bag-shaped structure 32 does not have the joint margins in the inner wall portion 41, the compression effective width for compressing the living body was the same as the width H of the bag-shaped structure 32.

As a comparative example, a bag-shaped structure 32 having joint margins 151 in the inner wall portion 141 was prepared as shown in FIG. 10(a). As the thermoplastic elastomer constituting this bag-shaped structure 132, thermoplastic polyurethane having the same characteristics as that used for the bag-shaped structure 32 of the example was used. The width H of the bag-shaped structure 32 was 25 mm, and the width of the joint margins 151 was 2 mm. Since the bag-shaped structure 32 has two joint margins 151 along the width direction, the compression effective width of the bag-shaped structure 132 for compressing the living body was 21 mm.

As an evaluation test, a blood vessel compression characteristic evaluation test was performed on each of the bag-shaped structures 32 and 132 of the example and the comparative example.

As the blood vessel pressure characteristics evaluation test, a blood pressure of the same person was actually measured by alternately using an upper arm blood pressure monitor, and blood pressure monitors 1 assembled from the bag-shaped structures 32 and 132 prepared in the example and comparative example, 10 times for each monitor.

Here, an upper arm blood pressure monitor model HEM-7120 manufactured by Omron Healthcare Co., Ltd. was used as the upper arm blood pressure monitor. The upper arm blood pressure monitor was attached to the upper arm, and the blood pressure monitors 1 of the example and the comparative example were attached to the wrist. Bag-shaped structure 32 of which standard deviation of the difference in the blood pressure values of 10 times was 7 mmHg or more was determined as defective because the blood vessel compression characteristics were poor and the measurement accuracy would be unstable, and the bag-shaped structure 32 of which standard deviation was less than 7 mmHg was determined as good because the blood vessel compression characteristics were good and the measurement accuracy would be stable.

As a result of the evaluation test, the bag-shaped structure 32 of the example had a standard deviation of 6 mmHg and was determined as good. On the other hand, the bag-shaped structure 132 of the comparative example had a standard deviation of 23 mmHg and was determined as defective.

These results show that, if the bag-shaped structure 32 used in the cuff 12 of the blood pressure monitor 1 has the configuration of the above-described embodiment or each modification, the bag-shaped structure 32 can provide high blood pressure measurement accuracy and provide the cuff 12 of the blood pressure monitor 1 with an appropriate function, even when the cuff width is reduced.

1. A bag-shaped structure used in a cuff for blood pressure measurement configured to be wrapped around a living body, inflate when a fluid is supplied to an internal space, and compress the living body, the bag-shaped structure comprising:
an inner wall portion provided on a living body side;
an outer wall portion facing the inner wall portion;
a pair of side wall portions bent toward the internal space and continuous with the inner wall portion and the outer wall portion, at least part of which is formed integrally with the inner wall portion.

2. The bag-shaped structure according to claim 1, wherein the inner wall portion, the outer wall portion, and the pair of side wall portions are molded from a thermoplastic elastomer.

3. The bag-shaped structure according to claim 1, wherein the pair of side wall portions each include: a part on the living body side with respect to an intermediate position in a direction in which the inner wall portion faces the outer wall portion, which is formed integrally with the inner wall portion; a part on another side with respect to the intermediate position in the direction in which the inner wall portion faces the outer wall portion, which is formed integrally with the outer wall portion; and a joint portion at the intermediate position where both parts are joined.

4. The bag-shaped structure according to claim 1, wherein the inner wall portion and the pair of side wall portions are integrally formed.

5. The bag-shaped structure according to claim 1, wherein the pair of side wall portions are each bent toward the internal space at a plurality of positions.

6. A method for manufacturing a bag-shaped structure used in a cuff for blood pressure measurement configured to be wrapped around a living body, inflate when a fluid is supplied to an internal space, and compress the living body, the bag-shaped structure comprising: an inner wall portion provided on a living body side; an outer wall portion facing the inner wall portion; and a pair of side wall portions bent toward the internal space, the method comprising:
forming a thermoplastic elastomer sheet into a sheet member in a shape of the inner wall portion and at least part of each of the pair of side wall portions;
trimming the formed sheet; and
joining the sheet member to another sheet member in a shape of other part of the side wall portion and the outer wall portion.

7. The method for manufacturing a bag-shaped structure according to claim 6, wherein the sheet member is formed by vacuum blow molding.

8. The method for manufacturing a bag-shaped structure according to claim 6, wherein
the sheet member and the other sheet member include joint margins for joining, and
the joint margins are joined in the joining of the sheet member and the other sheet member.

9. The method for manufacturing a bag-shaped structure according to claim 8, wherein the joining of the joint margins is performed by a high-frequency welder.
